(19) United States
(12) Patent Application Publication
(10) Pub. No.: US 2016/0249838 A1
(43) Pub. Date: Sep. 1, 2016

(54) Method and Apparatus for Effective Detection of Respiratory Blockage Using CO2 Monitor

(71) Applicants: Lawrence Cheng, San Jose, CA (US); Charles T. Cheng, San Jose, CA (US)

(72) Inventors: Lawrence Cheng, San Jose, CA (US); Charles T. Cheng, San Jose, CA (US)

(21) Appl. No.: 15/055,640

(22) Filed: Feb. 29, 2016

Related U.S. Application Data
(60) Provisional application No. 62/126,561, filed on Feb. 28, 2015.

(51) Int. Cl. A61B 5/145; A61B 5/00

(52) U.S. Cl. CPC A61B 5/14542; A61B 5/746

(57) ABSTRACT

A system and method are provided to detect a possible respiratory blockage by using personalized carbon dioxide (CO₂) concentration change patterns. The personalized change patterns can intelligently learn new change pattern to increase its accuracy. Advanced pattern recognition is used to detect abnormal CO₂ concentration change pattern by comparing to personalized patterns, and allowing the system to trigger alarm to alert a caregiver or a guardian.

### CROSS-REFERENCE

This application claims the benefit of U.S. Provisional Application with Ser. No. 62/126,561, filed on Feb. 28, 2015, the entire content is hereby incorporated by reference as if fully set forth herein, under 35 U.S.C. §119(e).

### FIELD OF INVENTION

This disclosure generally relates to systems for monitoring Carbon Dioxide (CO₂) in human's respiratory system for early detection of health problems.

### BACKGROUND

Currently, the third leading cause of death in homes across the United States is caused by choking and suffocation. Furthermore, over 3,300 individuals die every year from asthma attacks and there are 1.75 Million emergency room visits per year dealing with asthma attacks. In all of these situations, respiratory blockage occurs, and hazards such insufficient intake of oxygen and the removal of carbon dioxide from the blood result. In this case, if no help is quickly given the victim may suffer from dizziness, lack of consciousness, and death within minutes.

### SUMMARY

Various embodiments of methods and apparatus for detecting respiratory blockage are contemplated. In one embodiment, a system may detect diffused carbon dioxide (CO₂) concentration level by a biosensor from a person's skin while the person is engaging in an activity. The system analyzes changes of the CO₂ concentration level and generates a first CO₂ change pattern which is recorded as a first personalized pattern in a database. The systems can compare the first recorded personalized pattern to a newly generated CO₂ change pattern by detecting diffused CO₂ again from the person, and add the newly generated CO₂ change pattern as a second personalized pattern to the database if the first CO₂ change patter is substantially different from the newly generated CO₂ change pattern.

These and other embodiments will become apparent upon reference to the following description and accompanying drawings.

### BRIEF DESCRIPTION OF DRAWINGS

The present invention is illustrated by way of example, and not by way of limitation in the figures of the accompanying drawings and in which like reference numerals refer to similar elements and in which:

FIG. 1 is a generalized block diagram illustrating a CO₂ monitor system applied to human skin, according to an embodiment;

FIG. 2A illustrates a graph of CO₂ concentration level measured under different activities, according to an embodiment;

FIG. 2B illustrates another graph of CO₂ concentration level measured under different activities, according to an embodiment;

FIG. 3 is a flow chart illustrating steps for the operation of a monitor system, according to an embodiment;

FIG. 4 is a flow chart illustrating steps for the operation of a monitor system, according to an embodiment;

FIG. 5 is a flow chart illustrating steps for the operation of a monitor system, according to an embodiment.

### DETAILED DESCRIPTION

It is desirable to have a wearable, portable monitor that can monitor internal blood CO₂ levels of humans in order to prevent deaths caused by respiratory blockage, such as by choking or by asthma. Currently, there are no known device designs that allow continuous monitoring of the blood CO₂ levels of a patient or individual outside of an intensive care unit or by allowing individuals to partake in everyday activities, and this invention is aimed at solving this problem.

In the following description, numerous specific details are set forth to provide a thorough understanding of the methods and mechanisms presented herein. However, one having ordinary skill in the art should recognize that the various embodiments may be practiced without these specific details. In some instances, well-known structures, components, signals, computer program instructions, and techniques have not been shown in detail to avoid obscuring the approaches described herein. It will be appreciated that for simplicity and clarity of illustration, elements shown in the figures have not necessarily been drawn to scale. For example, the dimensions of some of the elements may be exaggerated relative to other elements.

Advantages of Measuring Arterial pCO₂

The analysis of the partial pressure of carbon dioxide in arterial blood, pCO₂, is of great significance in medical diagnosis because pCO₂ is an indicator of alveolus ventilation and the acid-base balance of the human body. In particular, the continuous monitoring of arterial pCO₂ is essential for surgical and serious patients who depend on an artificial ventilator. However, the blood-sampling method has restrictions, which makes it very invasive to patients, and needs much time for analysis because blood is directly obtained from the human body. In other cases of emergency in which patients require noninvasive analysis, arterial p CO₂, is inferred from end-tidal carbon dioxide, EtCO2, analysis, which does not accurately determine the arterial pCO2 and is very inconvenient because patients should breathe through a catheter. On the other hand, it is known that transcutaneous pCO2 measurement results agree with those of arterial blood pCO2 measurement, which is determined by blood-sampling method. Moreover, it has merits such as the arterial pCO2 in a capillary vessel can be measured in real time.

Basic Components of a CO₂ Monitor System

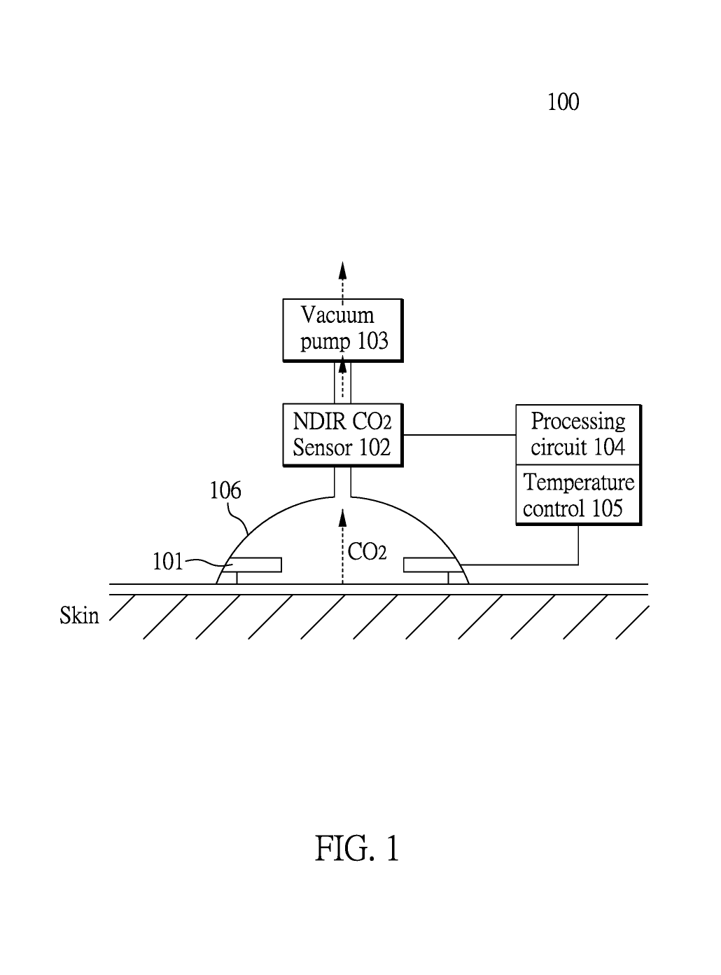

FIG. 1 illustrates a general block diagram of the basic components used for collecting and measuring diffused CO₂ from skin. In an embodiment, a CO₂ monitor system 100 comprises a skin-mount pad 101 warming the skin and capable of adjusting temperature between 37° C. and 42° C. to obtain diffused CO₂ gas. The warming pad 101 is inside a cover 106 and not directly in contact with skin, but could warm the skin to allow CO₂ diffusion. The CO₂ monitor system 100 also includes a Non-Dispersive Infrared (NDIR) CO₂ sensor 102 for measuring the pCO₂ level. NDIR CO₂ sensor 102 is used here for its sensitivity, accuracy and miniature size but other types of CO₂ sensor may be used as long as they can serve the same purpose. A vacuum pump 103 with adjustable flow rate is connected to a NDIR CO₂ sensor to move the collected diffused CO₂ gas to the CO₂ sensor for measurement. The circulation rate of vacuum pump should be close to the pCO₂ diffusion rate of skin. If the CO₂ sensor is sufficiently close to the skin to sense CO₂ gas quickly, the vacuum pump 103 may not be needed. Depending on the types of CO₂ sensor, a vacuum pump may or may not be used. A signal processing circuit 104 connected to NDIR CO₂ 102 sensor is used to continuously record and calculate the changing pattern of pCO₂ level and trigger an alarm when an unusual pattern occurs, a pattern that indicates a likelihood of respiratory blockage, such as choking or asthma attack. The alarm signal is transmitted to the guardian of an infant or a child or to a caregiver. The alarm may be an audio signal to alert nearby help or a wireless signal transmitted to guardian's or caregiver's mobile devices or emergency centers such as 911 or hospital. In another embodiment, skin-mount pad 101 with temperature control 105, vacuum pump 103, CO₂ sensor 102, signal processing circuit 104 and alarm (not shown) may be integrated together as one portable unit, such as a small electronic patch on skin, and be made waterproof.

Mechanism of Respiratory Blockage Detection

In order to trigger alarm for abnormal CO₂ changes, the detected CO₂ level does not have to be accurate in its absolute value but needs to be consistent. Therefore, a high temperature may not be necessary to obtain a large amount of diffused CO₂ gas, and thus a lower temperature of 40° C. or below may be sufficient. Consequently, the CO₂ monitor system can be worn by an individual for a considerable time or even 24 hours without causing any discomfort.

When a person is in healthy condition and behaving normally, the detected pCO₂ level should stay roughly the same. It may also move to a different level and then stabilize when the person engages in a different activity, such as dancing, walking or exercise. However, when a respiratory blockage occurs, the detected pCO₂ level would show a continuously changing pattern as the arterial CO₂ continues to build up, such as an upward trend. It is this changing pattern that helps to identify the early sign of respiration problem. Since the CO₂ change is more pronounced than the O₂ change when a person experiences a respiration problem, CO₂ detection would be more effective in achieving an early detection of the problem.

CO₂ Measurement and Data

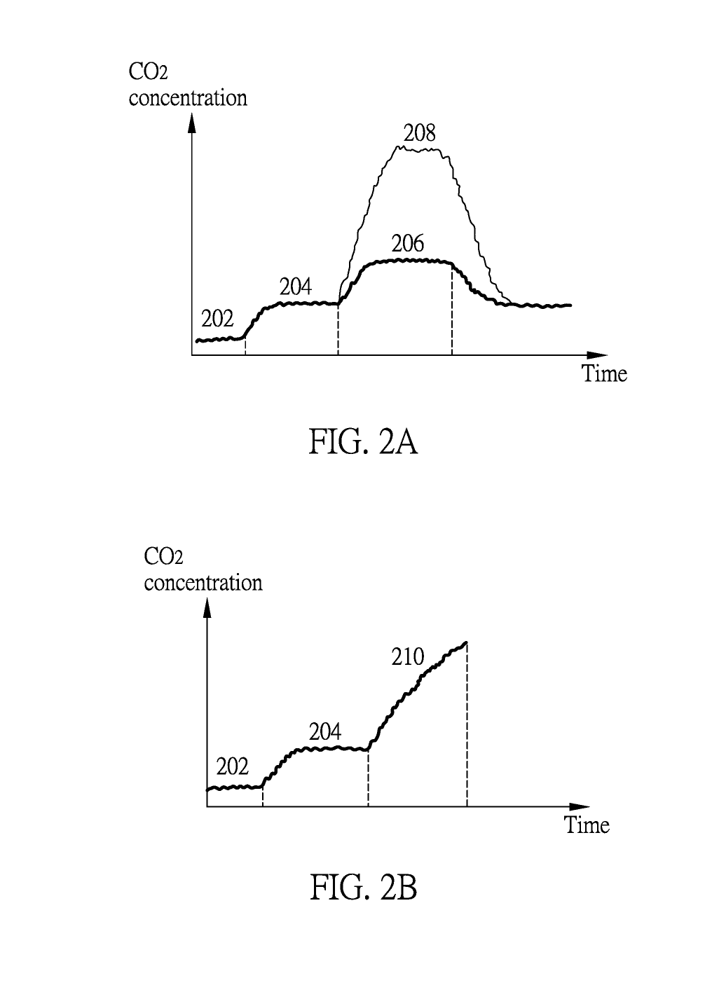

FIGS. 2A and 2B illustrate the change of CO₂ level in various conditions—normal and blockage of air passage. Normal condition may include quiet activity, mild activity and rigorous activity. In FIG. 2A, the CO₂ monitor system 100 is calibrated to open air as illustrated in section 202 of the graph. Once the CO₂ monitor system 100 is applied to a test subject (e.g. a person) in a way that does not obstruct the person's daily activity, such as wrist, arm or shoulder, the CO₂ level increases gradually and eventually stabilizes as shown in section 204. When the person engages in a mild activity that increase the heart rate such as walking, the CO₂ level changes again and then stabilizes as shown in section 206. Occasionally, when a person engages in an vigorous activity such as running or biking, the CO₂ level may jump in a short period of time and fall back to normal level when the heart rate decreases as shown in dash line of section 208.

In FIG. 2B, section 202 illustrates the calibration period to open air. When a person, the test subject, is in a quiet activity such as sitting and reading a book, the CO₂ level is stabilized as in section 204. When respiratory blockage occurs, (or the test subject holds his/her breath), the CO₂ level starts to change and continues to increase for a prolonged period of time, as in section 210, as long as the blockage condition is not cleared. When this changing pattern continues for a certain amount of time, an alarm may be triggered to notify guardians or caregivers.

Typically, the CO₂ concentration of calibrated value to open air is around 400 parts per million (ppm) at sea level as shown in section 202 of FIGS. 2A & 2B. When the CO₂ monitor system is applied to a human skin, the CO₂ concentration could rise up to a few thousand ppm (e.g. 1,000˜5,000 ppm) and stabilize as shown in section 204 of FIGS. 2A & 2B. However, when a person engages in a vigorous activity, the CO₂ concentration could increase a few thousand ppm in just tens of seconds and then stabilize as shown in section 208 of FIG. 2A. When a respiratory blockage occurs, CO₂ concentration may increase between tens to hundreds of ppm continuously for a long period of time depending on severity of the blockage condition as shown in section 210 of FIG. 2B.

The CO₂ concentration values in FIGS. 2A and 2B are for illustration only, not absolute values since these values may vary depending on person's health condition, measuring devices and environmental conditions. However, the general trends (or patterns) illustrated in FIGS. 2A and 2B are substantially the same and consistent for different persons.

In order to detect the changing pattern or trend of CO₂ level, a moving average may be used for this purpose. The moving average calculates an average value of a subset of data within a certain period of time or window, for example, the data collected within the past 2 seconds or 5 seconds. The calculation repeats on different subsets of data as the window moves (i.e. center of the subset changes). To make the CO₂ monitor system more sensitive to the change or shorter trend, the system can be configured to use a smaller window. Likewise, the CO₂ monitor system can be configured to use a larger window to detect longer trend.

Alternatively, the slopes of a changing curve of CO₂ level may be used. For example, the beginning slope and the ending slope of a curve together with the time between these two slopes may be used to recognize the trend of CO₂ level change. Typically, the CO₂ curve of a vigorous activity is steep and stabilizes within a short period of time. In addition to the previously discussed methods, other methods for detecting CO₂ level change may be contemplated.

Since each person may have unique CO₂ level patterns, the CO₂ monitor system may create a personalized database for each user and use advanced pattern recognition techniques to quickly detect an abnormal CO₂ level change. In one embodiment, the CO₂ monitor system may request a user to perform a few basic daily activities such as quite activity (e.g. lying still), mild activity (e.g. walking), and vigorous activity (e.g. running) for one to two minutes each. Then the system can record CO₂ level patterns associated with these activities for the particular user and store as a personalized database for future comparison. In another embodiment, the CO₂ monitor system may intelligently learn the user's daily activities through the user's interaction (e.g. when user indicates a false alarm) and categorize the corresponding CO₂ level patterns for future use, to be illustrated later.

Configuration Modes of CO₂ Monitor System

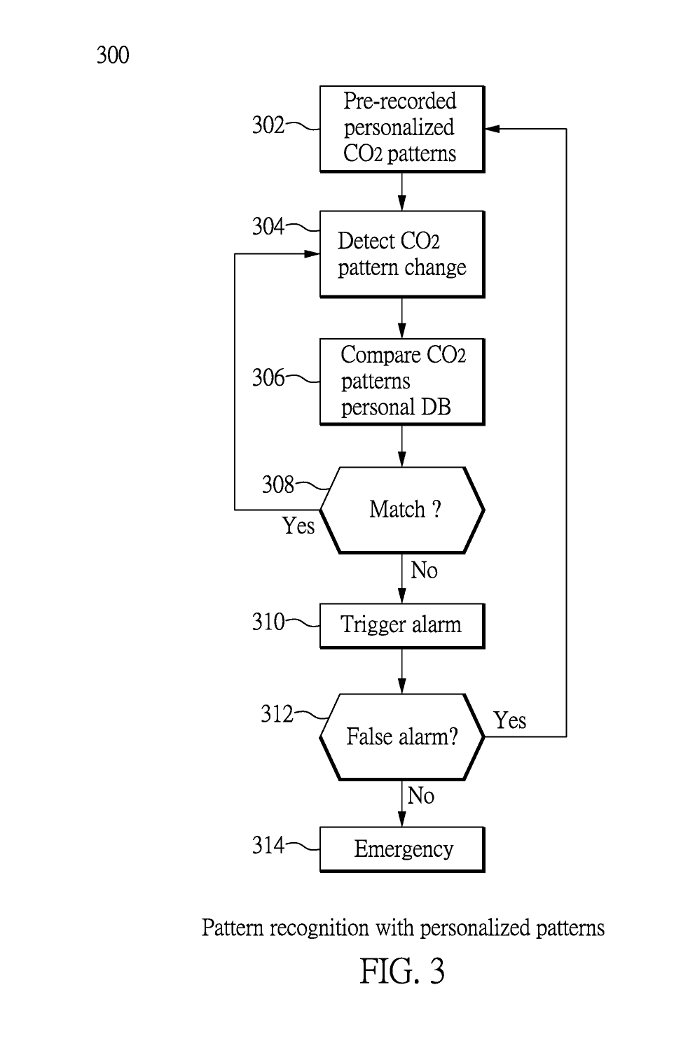

The CO₂ monitor system 100 may be configured to provide different levels of alerts when detecting a likely respiratory problem. In one embodiment, as shown in FIG. 3, the monitor may be configured to trigger an alarm for any abnormal change in CO₂ level for early warning and the warning level can be set to yellow color to indicate a possible concern. For example, when a CO₂ level pattern deviates from recorded basic personalized patterns in the database at any time, the system may trigger a warning. Once the guardian or caregiver confirms it is a false alarm, the system can learn from this pattern and add it to the personalized database.

In FIG. 3, at step 302, they system pre-recoreds personalized CO₂ patterns under different conditions and activities (e.g. quiet activity, mild activity and vigorous activity), as described earlier referring to FIG. 2A. At step 304, the system detects a CO₂ pattern change, then it compares the change to the pre-recorded personalized patterns stored in database as in step 306. At step 308, if the change pattern matches the personalized data, the system continues to monitor and detect any future pattern change. If the change pattern does not match the personalized data, the system triggers an alarm at step 310. At step 312, a caregiver or a guardian or the user can check whether it is a false alarm. If it is a false alarm, the user can simply acknowledge and allow the system to add the pattern to the personalized database. If it is not a false alarm, the emergency alarm has achieved its purpose at step 314. Since the recorded CO₂ pattern is a personalized data, the system should not trigger too many false alarm, and the accuracy increases dramatically after learning a few unusual patterns.

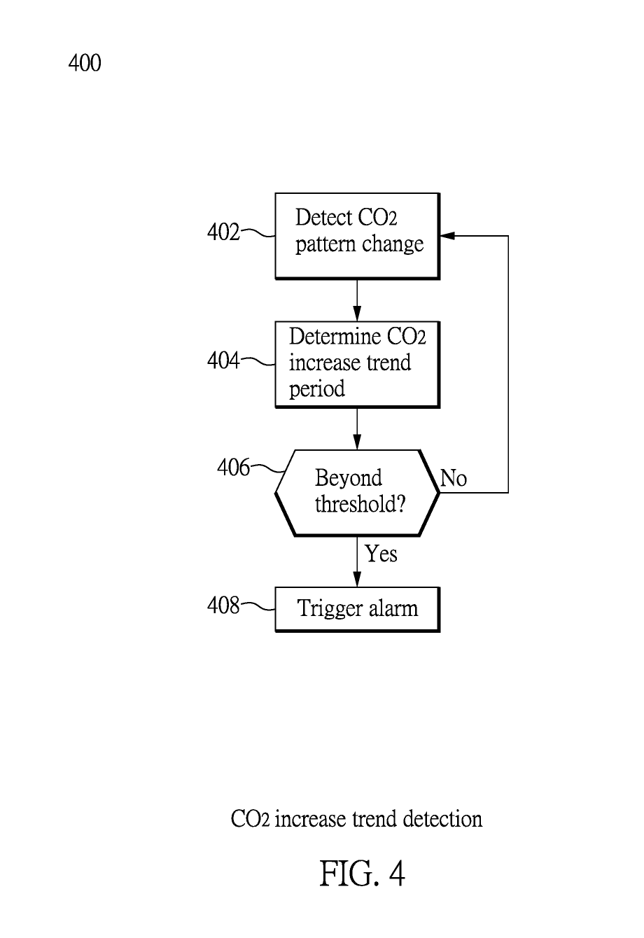

In another embodiment, as shown in FIG. 4, the monitor can be configured to trigger an alarm when it detects a trend of abnormal changes, for example a steady increase in moving average of CO₂ level beyond a certain period of time. Yet in another embodiment, the trend of change can be set to duration of 10 seconds, 20 seconds or 30 seconds, etc. Usually, such prolonged trend of CO₂ level change is a likely indicator of respiratory blockage and the alarm level can be set to red to indicate a severe condition for immediate attention. Yet in another embodiment, the CO₂ monitor system may record visual information of the CO₂ changing pattern for a certain period of time, so the guardian or caregiver can review the history that contributes to the trigger of an alarm.

In FIG. 4, at step 402, the CO₂ monitor system detects a pattern change. At step 404, the system can utilized one of the methods mentioned earlier to determine the CO₂ change trend within a certain period of time. At step 406, if the system detects the relation between the change trend and time duration is beyond a preset threshold, the system triggers an alarm at step 408. Otherwise, the system continue to monitor any CO₂ pattern change or deviation.

Consideration of Human Factors

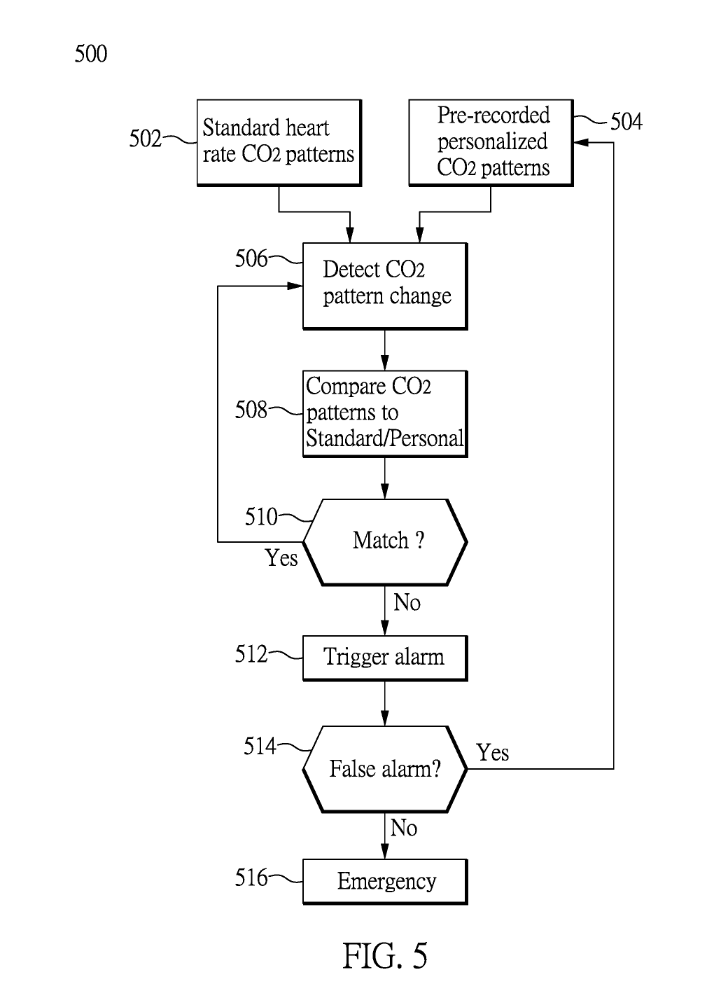

As discussed earlier, different human activities may create different CO₂ level and one factor that associates with these activities is the heart rate. Therefore, in one embodiment, the CO₂ monitor system may incorporate a heart rate sensor to help interpret CO₂ level changes besides checking personalized CO₂ pattern database. For example, when heart rate increases, decreases or stay constant, there will be corresponding changes in patterns of CO₂ level, which may be stored as standard patterns. If a detected CO₂ level pattern is different from expected patterns (either standard patterns or personalized patterns or both), it is likely that respiratory problem has occurred and an alarm may be triggered. FIG. 5 illustrates the use of both standard heart rate CO₂ patterns and personalized CO₂ patterns in CO₂ monitor system.

At step 502, the CO₂ monitor system incorporates some standard heart rate CO₂ patterns that normally occur in human body. At step 504, the system pre-records some personalized CO₂ patterns. By taking into account the heart rate change of the user to further supplement the personalized patterns, the system has further knowledge about the activities of the user and can select the appropriate pattern category, such as quiet, mild or vigorous category, for comparison. Alternatively, the system can separate these standard heart rate patterns and personalized patterns, and allows to select either one for use. At step 506, the system monitors and detects any CO₂ pattern changes in the user's body. At step 508, the system compares the monitored pattern to the hybrid patterns to detect any deviation. At step 510, if there is a match, the system continues to monitor body CO₂ pattern change. If there is a mismatch or substantial difference, the system triggers an alarm at 512. At step 514, a caregiver or a guardian or the user can check whether it is a false alarm. If it is a false alarm, the user can simply acknowledge and allow the system to add the pattern to the personalized database, as in step 504. If it is not a false alarm, the emergency alarm has achieved its purpose at step 516.

In addition to human activities, the easy use and comfort of a CO₂ monitor system are important to a wearer of such device. Besides miniaturizing the monitor system (or device) to allow a person to wear on any part of its body without noticing it, the comfort of using the device may affect a person's willingness. In one embodiment, the skin-mount pad may include several small heating apparatuses that can turn on and off alternatively to achieve constant temperature around the skin while reducing a prolonged heating of a particular spot on skin. For example, four small heating apparatuses may be used and each one may be turned on for an hour and rotate around these apparatus to avoid heating the same spot on wearer's skin.

Consideration of Environmental Factors

Since temperature and air pressure may affect CO₂ concentration level, some compensation mechanisms may be used. For example, the CO₂ monitor system may incorporate a temperature/pressure sensor which helps take environmental factors into account. The environmental factors may include ambient temperature change (e.g. room temperature), air pressure change (e.g. due to altitude), etc. For the purpose of consistency, in one embodiment, the CO₂ monitor system may have an adjustable heating apparatus that can sense the ambient temperature and adjust its heating temperature accordingly. For example, when ambient temperature is lower than 40° C., the heating apparatus can increase heating temperature to keep the temperature inside skin-mount pad close to 40° C. for sufficient CO₂ gas diffusion. However, when the ambient temperature is above 40° C., the heating apparatus may turn off and/or take into account the CO₂ level change due to higher ambient temperature when determining the CO₂ level pattern. Similarly, higher or lower air pressure may also change the CO₂ concentration level. However, the change is minor and the long term CO₂ level will stabilize and should not affect the detection of respiratory blockage.

In the foregoing description, embodiments of the invention have been described with reference to numerous specific details that may vary from implementation to implementation. The descriptions and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A method for detecting respiratory blockage, comprising:
detecting diffused carbon dioxide (CO₂) concentration level by a biosensor from a person's skin while said person is engaging in an activity;
analyzing changes of said CO₂ concentration level;
generating a first CO₂ change pattern based on said analyzed changes of said CO₂ concentration level;
recording said CO₂ change pattern of said person as a first personalized pattern in a database;
comparing said first recorded personalized pattern to a newly generated CO₂ change pattern by detecting diffused CO₂ from said person; and,
adding said newly generated CO₂ change pattern as a second personalized pattern to said database if the first CO₂ change patter is substantially different from said newly generated CO₂ change pattern.

2. The method of claim 1, wherein said activity is a quiet activity or a mild activity or a vigorous activity.

3. The method of claim 1, wherein said comparison triggers an alarm when the first CO₂ change patter is substantially different from said newly generated CO₂ change pattern.

4. The method of claim 1, wherein said adding is determined by said person.

5. The method of claim 1, further comprising adding a pre-recorded CO₂ change pattern of heart rate change to said database.
